(19) United States
(12) Patent Application Publication
(10) Pub. No.: US 2011/0313497 A1
(43) Pub. Date: Dec. 22, 2011

(54) Therapeutic and Thermoregulatory Devices with Aerogel Insulation

(76) Inventor: Brian MCFARLANE, Hubbard, NE (US)

(21) Appl. No.: 12/820,090

(22) Filed: Jun. 21, 2010

(51) Int. Cl. A61F 7/08; A61F 7/10; A61F 7/00

(52) U.S. Cl. 607/109; 607/114; 607/108; 607/111

(57) ABSTRACT

A device, combining a component of aerogel insulation with a heating or cooling component, for therapeutic, thermoregulatory, or therapeutic and thermoregulatory use with people or animals. Aerogels perform well as insulators for both high-temperature and low-temperature or cryogenic insulation. The aerogel insulation component and the heating and/or cooling component are enclosed in a structure that supports and retains the components. In some embodiments, a therapeutic garment includes an aerogel insulation layer and a heating and/or cooling system, both positioned between an inner layer and an outer layer. Some embodiments also include a middle layer separating the aerogel insulation layer from the heating and/or cooling system. In some embodiments, an aerogel insulation layer comprises a carrier material permeated with diffused fragments of aerogel material.

### CROSS-REFERENCE TO RELATED APPLICATIONS

Not Applicable

### STATEMENT REGARDING FEDERALLY-SPONSORED RESEARCH OR DEVELOPMENT

Not Applicable

### BACKGROUND OF THE INVENTION

1. Field of Invention

The present invention relates generally to thermal insulation materials and more particularly to thermal insulation materials for use in therapeutic and thermoregulatory devices.

2. Description of the Related Art

Many types of insulators for use in therapeutic and thermoregulatory garments and other devices are known in the art. Typical of the art in this field are the following:

| U.S. Patent or | Date of Issuance | |
|---|---|---|
| Publication No. | or Publication | Inventor(s) |
| 4,356,570 | Nov. 2, 1982 | Vernon et al. |
| 4,404,460 | Sep. 13, 1983 | Kerr |
| 4,569,087 | Feb. 11, 1986 | Kerwin |
| 4,951,317 | Aug. 28, 1990 | Gray et al. |
| 4,985,934 | Jan. 22, 1991 | Perry |
| 5,035,001 | Jul. 30, 1991 | Novick |
| 5,201,075 | Apr. 13, 1993 | Svetich |
| 6,052,824 | Apr. 25, 2000 | May |
| 6,138,664 | Oct. 31, 2000 | Hanada et al. |
| 7,494,946 | Feb. 24, 2009 | Gibson |
| 7,496,969 | Mar. 3, 2009 | Pieczynski |
| 2004/0111782 | Jun. 17, 2004 | Lenormand |
| 2004/0163154 | Aug. 26, 2004 | Cooper |
| 2005/0247365 | Nov. 10, 2005 | Schwartz |
| 2006/0213895 | Sep. 28, 2006 | Dennis |
| 2006/0254088 | Nov. 16, 2006 | McCormick |
| 2007/0077842 | Apr. 5, 2007 | Gibson et al. |
| 2007/0095808 | May 3, 2007 | Lacy |
| 2007/0118950 | May 31, 2007 | Hogge |
| 2008/0067163 | Mar. 20, 2008 | Axinte et al. |
| 2008/0197126 | Aug. 21, 2008 | Bourke et al. |
| 2009/0000002 | Jan. 1, 2009 | Hadash |

### BRIEF SUMMARY OF THE INVENTION

Disclosed is a device, combining a component of aerogel insulation with a heating or cooling component, for therapeutic, thermoregulatory, or therapeutic and thermoregulatory use with people or animals. Aerogels perform well as insulators for both high-temperature and low-temperature, or cryogenic, insulation. As used herein, in some embodiments aerogels include porous cross-linked polymers fabricated by causing monomers in a gel solution to react with each other to form a collection, or sol, of colloidal clusters, with the gel being subsequently replaced with air or other gas to leave a porous, low-density lattice. This most common process by which aerogels are fabricated is known in the art as the sol-gel process.

As disclosed herein, some embodiments of a therapeutic garment include an aerogel insulation layer and a heating and/or cooling system. In some embodiments, the aerogel insulation layer and a heating and/or cooling system are positioned between an outer layer and an inner layer. Some embodiments include a middle layer separating the aerogel insulation layer from the heating and/or cooling system. In some embodiments, the heating and/or cooling system is in direct contact with the skin of the human or animal user. Further, in some embodiments the aerogel insulation layer comprises a carrier material permeated with diffused fragments of aerogel material, thereby providing structural support and protection to the friable aerogel while still allowing the therapeutic garment to take advantage of insulative properties of the aerogel.

A therapeutic garment as described herein has many applications, including but not limited to uses as a jacket or a sleeve, as for baseball pitchers or tennis players; as a leg warmer for use on the leg; or as a wrap for the leg of a horse or other animal. Additional modifications and embodiments will be readily apparent to those skilled in the art.

### BRIEF DESCRIPTION OF THE DRAWINGS

The above-mentioned features of the invention will become more clearly understood from the following detailed description of the invention read together with the drawings in which:

FIG. 1 is a block diagram representation of one embodiment of the invention;

FIG. 2 is a section view of one embodiment of the invention;

FIG. 3 is perspective view of one embodiment of the invention, shown as worn by a human being;

FIG. 4 is a frontal view of the embodiment shown in FIG. 3;

FIG. 5 is a section view of the embodiment shown in FIGS. 3 and 4;

FIG. 6 is a perspective view of one embodiment of the invention;

FIG. 7A is a front view of the embodiment shown in FIG. 6, showing primarily the outer layer of the garment;

FIG. 7B is a back view of the embodiment shown in FIG. 6 and FIG. 7A, showing primarily the inner layer of the garment;

FIG. 8 is a section view of the embodiment shown in FIG. 6, FIG. 7A and FIG. 7B;

FIG. 9 is a perspective view of one embodiment of the invention for use on a horse's leg;

FIG. 10 is another view of the embodiment shown in FIG. 9;

FIG. 11 is a section view of the embodiment shown in FIG. 9 and FIG. 10;

FIG. 12 is a perspective view of one embodiment of the invention;

FIG. 13 is a perspective view of the embodiment shown in FIG. 12; and

FIG. 14 is a section view of the embodiment shown in FIGS. 12 and 13.

### DETAILED DESCRIPTION OF THE INVENTION

A device, combining a component of aerogel insulation with a heating or cooling component, for therapeutic, thermoregulatory, or therapeutic and thermoregulatory use with people or animals is disclosed.

FIG. 1 shows a block diagram representation of one embodiment of the present invention. In this embodiment, the device 101 includes an aerogel insulation component 102 and a heating and/or cooling component 104. Both the aerogel insulation component 102 and the heating and/or cooling component 104 are enclosed in a structure 105 that supports and retains the components. Further, some embodiments include an optional controller 114 in communication with the heating and/or cooling component 104 and adapted to allow the temperature within the device 101 to be controlled. Some embodiments include an optional thermostat 115 in communication with the heating and/or cooling component 104 and adapted to allow the temperature within the device 101 to be controlled.

In some embodiments the aerogel insulation includes porous cross-linked polymers fabricated according to the sol-gel process, with monomers in a gel solution being reacted with each other to form a collection, or sol, of colloidal clusters, and with the gel being subsequently replaced with air or other gas to leave a porous, low-density lattice. Many different types of aerogels are known in the art, including silica-based aerogels, carbon-based aerogels, and aerogels based on aluminum oxide, agar, sulfur, selenium, and other materials. In some embodiments of the present invention, the aerogels used in therapeutic garments exhibit high surface areas (approximately 400-1000 m²/g), high porosity (generally at least 50% porosity, and in many cases at least 95% porosity), large pore volume (approximately 3.8 mL/g or higher), and low bulk densities (generally less than 0.3 g/cm³). In many embodiments, and especially in embodiments with carbon-based aerogels, the aerogel includes pores with a pore diameter of less than 100 nm.

Although porous and filled with gas, aerogels inhibit convective heat transfer because air cannot circulate through the aerogel lattice. At the same time, aerogels inhibit conductive heat transfer because the gases that fill much of their volume are poor heat conductors. In some applications, the material used as the solid component of the aerogel lattice, such as silica, is also a poor heat conductor, thereby enhancing the aerogel's insulative performance. Further, many aerogels are efficient radiative insulators because the material used as the solid component of the aerogel lattice is itself an absorbent of infrared radiation or other wavelengths. Carbon atoms in carbon-based aerogels, for example, absorb infrared radiation, thereby inhibiting the transfer of thermal radiation.

Some aerogels include a mixture of solid materials to take advantage of the complimentary insulation-enhancing physical properties of different materials. For example, an aerogel formed from a colloidal mixture of silica and carbon combines the poor heat-conducting properties of silica with the infrared-absorbing properties of carbon, making a silica-carbon aerogel an effective insulating material. Those of skill in the art will appreciate that, in a silica-carbon aerogel, the ratio of silica to carbon in the colloidal mixture and the resulting lattice varies depending upon the properties sought in and the intended use of the particular aerogel. Those of skill in the art will also recognize that other mixtures and other composite aerogels are possible and are known in the art.

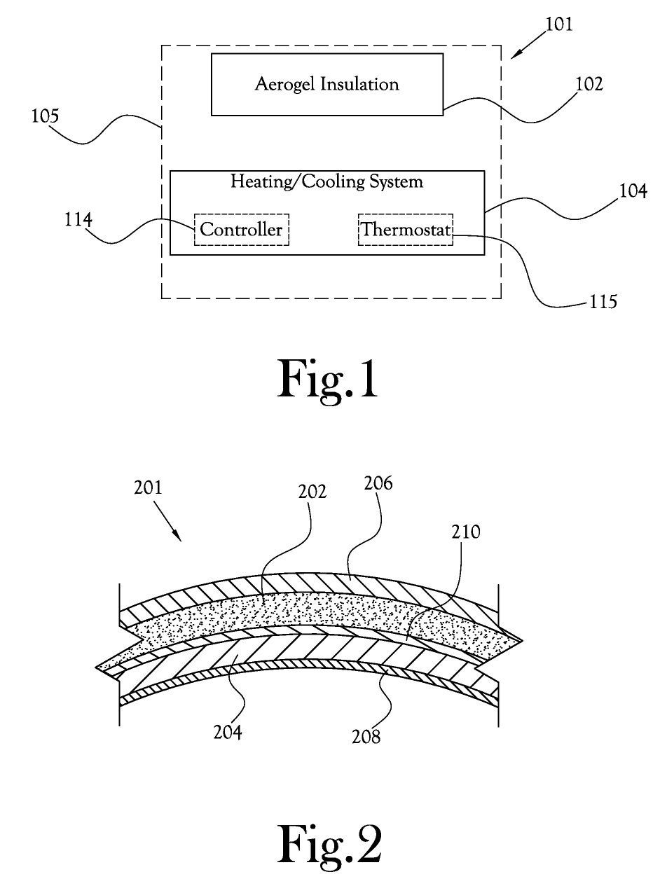

FIG. 2 shows a section view of one embodiment of the present invention. A therapeutic garment 201 includes an aerogel insulation layer 202 and a heating and/or cooling system 204, both positioned between an outer layer 206 and an inner layer 208. Some embodiments also include a middle layer 210 separating the aerogel insulation layer 202 from the heating and/or cooling system 204. The inner layer 208, during use of the garment, will be directed toward the human or animal subject and often will be in physical contact with the skin of the human or animal user; in some other embodiments, an additional layer, such as a moisture wicking or an antimicrobial base layer, will be interposed between the user's skin and the inner layer 208. Thus, the inner layer 208 is fabricated from a material that does not irritate the subject's skin and that allows for the passage of heat between the subject's body and the heating and/or cooling system 204. In some embodiments, the inner layer 208 is fabricated from a knitted or woven polyester material, or from a similar polymeric material. The outer layer 206, which during use of the garment will be oriented toward the air and will be positioned between the air and the aerogel insulation layer 202, is fabricated from a material adapted to protect the aerogel insulation layer 202 from exposure to the elements. In some embodiments, the outer layer 206 is fabricated from a knitted or woven polyester material, or from a similar polymeric material.

In some embodiments, the aerogel insulation layer 202 comprises a carrier material permeated with diffused fragments of aerogel material. In many embodiments, diffusing the aerogel within a carrier material provides structural support and protection to the friable aerogel while still allowing the therapeutic garment to take advantage of insulative properties of the aerogel. Further, diffusing the aerogel within a carrier material allows the aerogel insulation layer to be available in the form of a continuous sheet or roll that is amenable to conventional textile cutting and fabrication techniques, including the use of a die cutting machine. Thus, diffusing the aerogel within a carrier material in many cases enhances the ease with which the aerogel insulation layer is incorporated into the therapeutic garment during the fabrication of the therapeutic garment. Also, diffusing the friable aerogel within a carrier material in many embodiment reduces the production of dust from the aerogel—dust being a common drawback of the use of aerogels, a drawback that has prevented their use in many applications to date.

In some embodiments the carrier material is a polymeric material suitable for supporting and holding diffused aerogel fragments; in some embodiments, the carrier material includes fabrics or materials such as a polyester, polyethylene, or polyurethane substance. In some embodiments, the carrier material is a carbon felt or a similar carbon-based fibrous material suitable for supporting and holding diffused aerogel fragments. In some embodiments, the aerogel is directly adhered or attached to the carrier material. In some embodiments, the carrier material is a gel within which the diffused aerogel fragments are suspended.

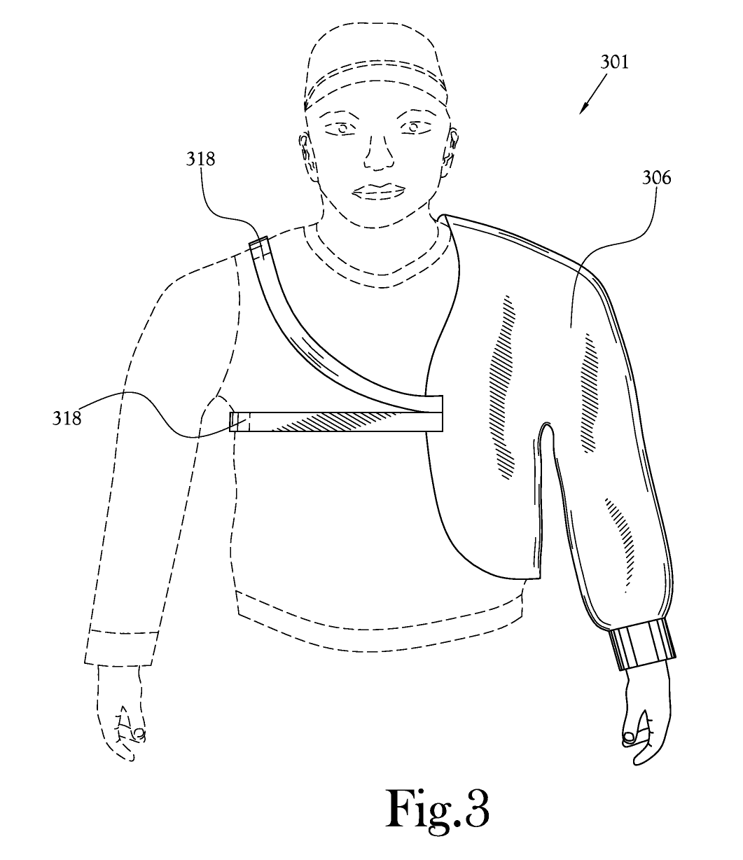

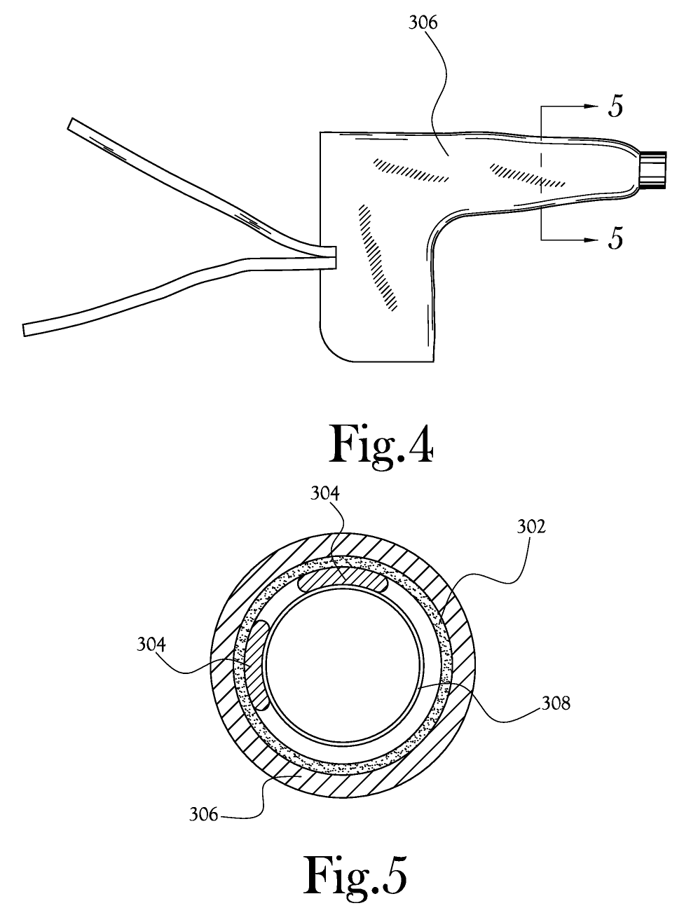

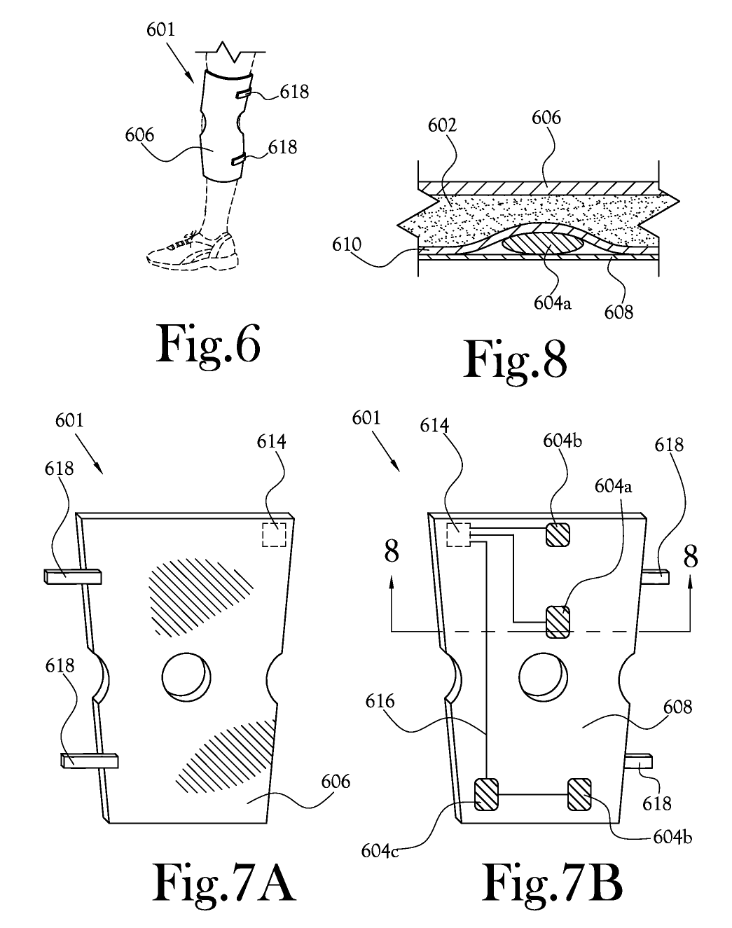

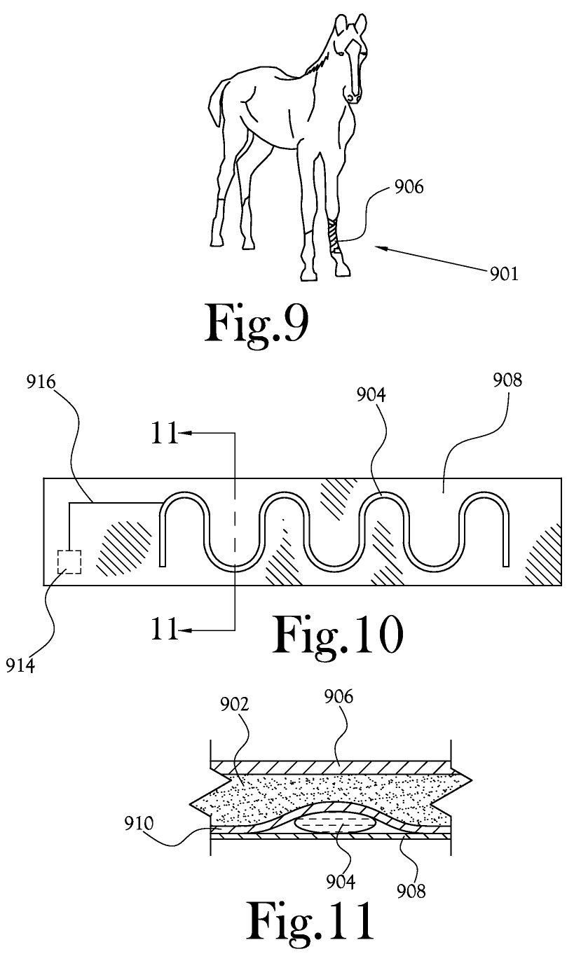

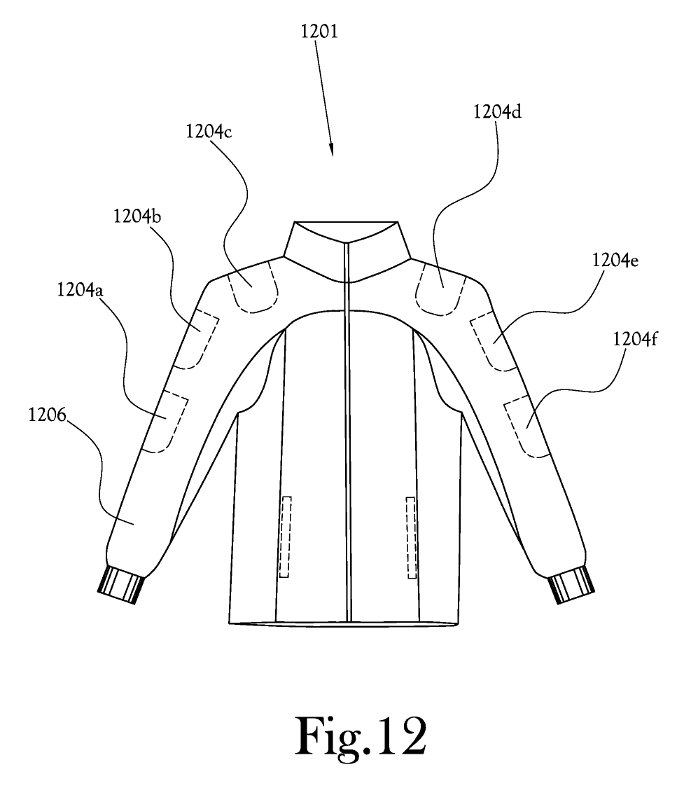

Embodiments for various applications are illustrated in FIGS. 3-11. Specifically, FIGS. 3-5 show a sleeve including aerogel insulation and a heating and/or cooling system; FIGS. 6-8 show a garment for a human leg including aerogel insulation and a heating and/or cooling system; FIGS. 9-11 show a garment for a horse's leg including aerogel insulation and a heating and/or cooling system; and FIGS. 12-14 show a jacket including aerogel insulation and a heating and/or cooling system.

FIGS. 3-5 show a sleeve 301 including aerogel insulation and a heating and/or cooling system. As shown in the section view of FIG. 5, the sleeve 301 includes an outer layer 306 and an inner layer 308. Between the outer layer 306 and the inner layer 308 are an aerogel insulation layer 302 and a heating and/or cooling system with heating and/or cooling elements 304. In one embodiment, the heating and/or cooling elements 304 are resistive heating elements connected to and drawing power from a power source. In some embodiments, the power source for the resistive heating elements is a portable electric power source. In one embodiment, the heating and/or cooling elements include removable oxygen-activated heat packs. In one embodiment, the heating and/or cooling elements include removable microwaveable heat packs. In one embodiment, the heating and/or cooling elements include removable ice packs. In one embodiment, the heating and/or cooling elements include removable cooling packs. In some embodiments, the heating and/or cooling elements include one or more packs with a liquid that is adapted to be either heated or cooled. In one embodiment, the heating and/or cooling elements include heat pipes that carry heated fluid to warm the sleeve. In one embodiment, the heating and/or cooling elements include passive heat dissipation elements, such as heat pipes, which carry heat away from the sleeve. In a further embodiment, the heating and/or cooling system includes a fan unit to dissipate heat carried by the heat pipes. In one embodiment, the heating and/or cooling elements include active cooling lines. The active cooling lines generally carry a reduced temperature fluid into the sleeve, where conductive heat exchange between the user's body and the active cooling lines. Following the heat exchange, the fluid at the elevated temperature is carried away from the sleeve, and the heat is dissipated. In one embodiment, the heating and/or cooling elements include a cooling element in which cooling is achieved using a Peltier cooler. In a further embodiment, the heating and/or cooling system includes any combination of heating and/or cooling elements. Further, in some embodiments the sleeve includes a controller in communication with the heating and/or cooling system that allows an operator to selectively power only one or only a select number of heating and/or cooling elements at a given time, so that the sleeve 301 is heating or cooling some muscle areas and not others. In some embodiments the heating and/or cooling system includes one or more thermostats that allow the temperature within the sleeve 301 to be controlled. Some embodiments of the invention include straps 318 or other devices for securing the sleeve 301 on a user, as shown in FIG. 3.

FIGS. 6-8 show a garment for a human leg including aerogel insulation and a heating and/or cooling system. As shown in FIGS. 7A and 7B and in the section view of FIG. 8, the garment 601 includes an outer layer 606 and an inner layer 608. Between the outer layer 606 and the inner layer 608 are an aerogel insulation layer 602 and a heating and/or cooling system. As shown in FIG. 8, some embodiments include a separating layer 610 between the aerogel insulation layer 602 and the heating and/or cooling system. In some embodiments, the heating and/or cooling system includes multiple electric heaters 604a-d, with each heater 604a-d connected to and drawing power from a power source 614 through power cables 616. In some embodiments, the heaters 604a-d are positioned on the garment 601 so as to be positioned over specific muscles when the garment is worn. Further, in some embodiments the power source 614 includes a controller that allows an operator to selectively power only one or only a select number of heaters 604a-d at a given time, so that the garment 601 applies heat to some muscle areas and not to others. In other embodiments of the garment 601, the heating and/or cooling system includes heating and/or cooling elements similar to those described above for the sleeve 301, including, in various embodiments, heat pipes that carry heated fluid, passive heat dissipation elements such as heat pipes, active cooling lines, and a Peltier cooler. In a further embodiment, the heating and/or cooling system includes any combination of heating and/or cooling elements. In some embodiments the heating and/or cooling system includes one or more thermostats that allow the temperature within the garment 601 to be controlled. Some embodiments also include devices 618 for securing the garment 601 to the leg during use.

FIGS. 9-11 show a leg wrap for a horse's leg including aerogel insulation and a heating and/or cooling system. As shown in FIG. 10 and in the section view of FIG. 11, the wrap 901 includes an outer layer 906 and an inner layer 908. Between the outer layer 906 and the inner layer 908 are an aerogel insulation layer 902 and a heating and/or cooling system 904. Some embodiments also include a separating layer 910 between the insulation layer 902 and the heating system 904. In the illustrated embodiment, as shown in FIG. 10, the heating and/or cooling system 904 includes a long tube substantially filled with a fluid. A power source 914 in communication with the heating tube 904 through a power cord 916 is adapted to heat the liquid in the heating tube 904 to provide therapeutic heat to the horse's leg. In other embodiments of the wrap 901, the heating and/or cooling system includes heating and/or cooling elements similar to those described above for the sleeve 301 and the garment for a human leg 601, including, in various embodiments, resistive heating elements, passive heat dissipation elements such as heat pipes, active cooling lines, and a Peltier cooler. In a further embodiment, the heating and/or cooling system includes any combination of heating and/or cooling elements. In some embodiments the heating and/or cooling system includes a controller that allows the temperature within the wrap 901 to be controlled. In some embodiments the heating and/or cooling system includes one or more thermostats that allow the temperature within the wrap 901 to be controlled. Those of skill in the art will recognize that the present invention could also be used for other animals, including (but not limited to) leg wraps and body wraps for dogs, or heated jackets for waterfowl hunting dogs.

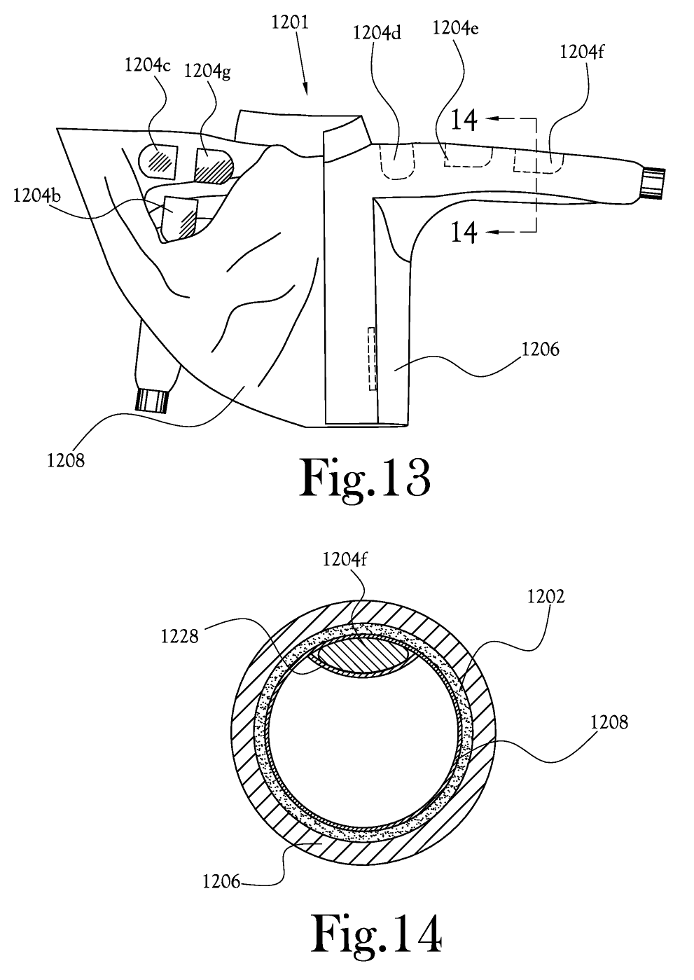

FIGS. 12-14 show a jacket 1201 including aerogel insulation and a heating and/or cooling system. As shown in the view of FIG. 13 and in the section view of FIG. 14, the jacket 1201 includes an outer layer 1206 and an inner layer 1208. Between the outer layer 1206 and the inner layer 1208 is an aerogel insulation layer 1202. The jacket 1201 also includes multiple heating and/or cooling elements 1204a-g. In the illustrated embodiment, the heating and/or cooling elements 1204a-g include multiple removable heating or cooling packs. As shown in the section view in FIG. 14, each heating or cooling pack is held in a pocket 1228 attached to the insulation layer 1202 or built into the inner layer 1208. In one embodiment, the heating and/or cooling elements 1204 are resistive heating elements connected to and drawing power from a power source. In one embodiment, the heating and/or cooling elements include removable oxygen-activated heat packs. In one embodiment, the heating and/or cooling elements include removable microwaveable heat packs. In one embodiment, the heating and/or cooling elements include removable ice packs. In one embodiment, the heating and/or cooling elements include removable cooling packs. In some embodiments, the heating and/or cooling elements include one or more packs with a liquid that is adapted to be either heated or cooled. Still other embodiments include heating packs, cooling packs, or heating and/or cooling packs that are not removable. In other embodiments of the jacket 1201, the heating and/or cooling system includes heating and/or cooling elements similar to those described above for the sleeve 301, the garment for a human leg 601, and the wrap 901, including, in various embodiments, resistive heating elements, passive heat dissipation elements such as heat pipes, active cooling lines, and a Peltier cooler. In a further embodiment, the heating and/or cooling system includes any combination of heating and/or cooling elements. In some embodiments the heating and/or cooling system includes a controller that allows the temperature within the jacket 1201 to be controlled. Further, in some embodiments the jacket 1201 includes a controller in communication with the heating and/or cooling system that allows an operator to selectively power only one or only a select number of heating and/or cooling elements at a given time, so that the jacket 1201 heats or cools some muscle areas and not others. In some embodiments the heating and/or cooling system includes one or more thermostats that allow the temperature within the jacket 1201 to be controlled.

While the present invention has been illustrated by description of several embodiments and while the illustrative embodiments have been described in detail, it is not the intention of the applicant to restrict or in any way limit the scope of the appended claims to such detail. Those familiar with the art will recognize that other embodiments of the present invention exist, including, but not limited to, garments for warming and/or cooling a hand or hands; garments for warming and/or cooling a foot or feet; garments for warming and/or cooling the head; a wrap or other garment for warming and/or cooling the kidneys; and wraps or other garments for warming and/or cooling specific muscles, organs, or body parts.

While the present invention has been illustrated by description of several embodiments, and while the illustrative embodiments have been described in detail, it is not the intention of the applicant to restrict or in any way limit the scope of the appended claims to such detail. Additional modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

1. A therapeutic garment for regulating the temperature of a body part comprising:
a layer of insulation, said insulation including an aerogel material, and
a heating and/or cooling system adapted to modify the temperature of the body part, said heating and/or cooling system being positioned substantially between said layer of insulation and the body part when said therapeutic garment is in use.

2. The therapeutic garment of claim 1 wherein said heating and/or cooling system includes at least one electric heating and/or cooling element.

3. The therapeutic garment of claim 2 further comprising a controller in communication with said electric heating and/or cooling elements, wherein each said electric heating element is independently controlled by said controller.

4. The therapeutic garment of claim 1 wherein said heating and/or cooling system includes an electric heater.

5. The therapeutic garment of claim 1 wherein said heating and/or cooling system includes removable packs adapted to be heated.

6. The therapeutic garment of claim 1 wherein said heating and/or cooling system includes removable packs adapted to be cooled.

7. The therapeutic garment of claim 1 wherein said heating and/or cooling system includes a liquid adapted to be heated.

8. The therapeutic garment of claim 1 wherein said heating and/or cooling system includes a liquid adapted to be cooled.

9. The therapeutic garment of claim 1 wherein said layer of insulation includes a carrier material adapted to hold diffused fragments of aerogel.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. The therapeutic garment of claim 1 wherein said therapeutic garment is adapted for use on a human body part.

22. The therapeutic garment of claim 21 wherein the human body part is selected from the group consisting of the shoulder, arm, elbow, wrist, hand, thigh, leg, knee, ankle, foot, head, neck, and torso.

23. The therapeutic garment of claim 1 wherein said therapeutic garment is adapted for use on a non-human animal.

24. The therapeutic garment of claim 23 wherein the non-human animal is selected from the group consisting of a horse and a dog.

25. A therapeutic device for regulating the temperature of a body part comprising:
a layer of insulation, said insulation including an aerogel material,
a temperature regulation system adapted to alternatively heat or cool the body part, said temperature regulation system being positioned substantially between said layer of insulation and the body part when said therapeutic garment is in use,
an outer layer adapted to protect said layer of insulation,
an inner layer positioned substantially between said temperature regulation system and the body part when said therapeutic garment is in use, and
a separating layer positioned substantially between said layer of insulation and said temperature regulation system.

26. The therapeutic garment of claim 25 wherein said temperature regulation system includes multiple temperature regulation elements.

27. The therapeutic garment of claim 26 further comprising a controller in communication with said electric heating element, wherein each said electric heating element is independently controlled by said controller.

28. The therapeutic garment of claim 25 wherein said layer of insulation includes a carrier material adapted to hold diffused fragments of aerogel.

29. The therapeutic garment of claim 25 wherein said therapeutic garment is adapted for use on a human body part.

30. The therapeutic garment of claim 29 wherein the human body part is selected from the group consisting of the shoulder, arm, elbow, wrist, hand, thigh, leg, knee, ankle, foot, head, neck, and torso.

31. The therapeutic garment of claim 25 wherein said therapeutic garment is adapted for use on a non-human animal.
